(19) United States
(12) Patent Application Publication
(10) Pub. No.: US 2024/0366436 A1
(43) Pub. Date: Nov. 7, 2024

(54) Elastic Laminate

(71) Applicant: Aplix, Le Cellier (FR)

(72) Inventors: Nathalie Christine Michelle Marie Moinard, Le Cellier (FR); Thierry Jacques Alfred Marche, Le Cellier (FR)

(21) Appl. No.: 18/689,163

(22) PCT Filed: Oct. 17, 2022
(86) PCT No.: PCT/EP2022/078871

§ 371 Date: Mar. 5, 2024

(30) Foreign Application Priority Data
Oct. 19, 2021 (FR) FR2111089

(51) Int. Cl. A61F 13/49; A61F 13/15; A61F 13/56; A61F 13/62

(52) U.S. Cl. CPC A61F 13/49012; A61F 13/15699; A61F 13/4902; A61F 13/5655; A61F 13/622; A61F 2013/15406; A61F 2013/49022; A61F 2013/49033

(57) ABSTRACT

Elastic laminate, extending widthwise in a first direction, specifically CD, and lengthwise in a second direction, specifically MD, including at least one nonwoven layer and at least one elastic film fixed by an upper face or lower face, respectively, to a lower face or upper face, respectively, of the at least one nonwoven layer; characterized in that, when the laminate is not in the stretched state, the nonwoven layer includes, in transverse cross-section according to the first direction, specifically in the transverse cross-section CD, curved sections in an inverted U, specifically in a Ω, separated from each other by intermediate sections.

The present invention relates to an elastic laminate which is intended to be used in the field of hygiene, and in particular for nappies or adult incontinence pants, the laminate extending in accordance with a given width corresponding to the direction CD (Cross Direction or Transverse Direction) and a large length corresponding to the unwinding direction during the manufacture of the laminate, referred to as the direction MD (Machine Direction), and comprising one stack of a nonwoven layer and at least one elastic film extending over a width that is less than or equal to said given width, in particular two lower and upper layers of nonwoven sandwiching the at least one elastic film. These laminates are particularly intended for use in the manufacture of elastic lugs intended to carry hooks and to be fixed to the edges of the rear central part of the waist of a nappy to engage with the loop elements originating from the front part of the waist to create a movable and elastically adjustable closure for the nappy.

A laminate of this type is known for example from document EP-A-1783257, in the name of the applicant.

Although this elastic laminate from the prior art has considerable advantages with respect to that which existed previously, it is desirable to improve it further, and in particular to increase its user comfort, in particular its apparent capacity, when it is in the form of an elastic lug of a waist of the nappy, for being stretched without the user having the impression that the laminate will break due to the stretching. It would also be desirable to improve its softness to the touch in the stretched state.

The present invention thus relates, according to a first aspect, to an elastic laminate, extending widthwise in a first direction, specifically CD, and lengthwise in a second direction, specifically MD, comprising:
at least one nonwoven layer; and
at least one elastic film fixed by an upper face or lower face, respectively, to a lower face or upper face, respectively, of the at least one nonwoven layer;
at least one adhesive layer, in particular glue, being provided between the at least one nonwoven layer and the at least one elastic film,
characterised in that
when the laminate is not in the stretched state, in particular before a first elongation of the laminate in the first direction, in particular before a first elongation in use once integrated in an absorbent element such as a nappy, the nonwoven layer comprises undulations which, in transverse cross-section according to the first direction, specifically in transverse cross-section CD, form curved sections in an inverted U, specifically in a Ω, separated from each other by intermediate sections,
the intermediate sections being in contact with the at least one adhesive layer, while the curved sections are not in contact with the at least one adhesive layer.

The present invention also relates, according to the first aspect, to an elastic laminate, extending widthwise in a first direction, specifically CD, and lengthwise in a second direction, specifically MD, comprising:
at least one nonwoven layer; and
at least one elastic film fixed by an upper face or lower face, respectively, to a lower face or upper face, respectively, of the at least one nonwoven layer;
the at least one nonwoven layer being fixed to the at least one elastic film in fixing zones, in particular by ultrasonic welding, calendering or laminating, in particular hot,
characterised in that
when the laminate is not in the stretched state, in particular before a first elongation of the laminate in the first direction, in particular before a first elongation in use once integrated in an absorbent element such as a nappy, the nonwoven layer comprises undulations which, in transverse cross-section according to the first direction, specifically in transverse cross-section CD, form curved sections in an inverted U, specifically in a Ω, separated from each other by intermediate sections,
the intermediate sections being in contact with the at least one elastic film, while the curved sections are not in contact with the at least one elastic film.

Preferably, the undulations extend, in the second direction, specifically MD, over the entire length of the at least one nonwoven layer, in particular over the entire length of the laminate, the length of the laminate being in particular greater than 20 mm, in particular greater than 30 mm, more particularly greater than 40 mm, even more particularly greater than 60 mm.

Preferably, the nonwoven layer or layers forming the at least one nonwoven layer extend(s) in the first direction over a total nonwoven width, in particular in the direction CD, and the elastic film or films forming the at least one elastic film extend(s) over a total elastic width, the ratio equal to the total elastic width to the total nonwoven width being between 0.3 and 0.9, preferably between 0.4 and 0.8.

Preferably, the curved sections extend in the first direction over a total curved section width, in particular in the direction CD, and the intermediate sections extend over a total intermediate section width, the ratio equal to the total curved section width to the total width of the nonwoven being between 0.25 and 0.85, preferably between 0.35 and 0.75.

Preferably, according to another aspect of the invention itself forming an invention, independently of the other aspects of the invention, and in particular independently of the first aspect, but which can also be implemented in a favourable manner in combination with each of these other aspects, and in particular with the first aspect,
the laminate having an initial thickness (e₀) before its first stretching, and when the laminate is stretched according to the first or the second direction up to a so-called maximum elongation, at least equal to 50%, the curve giving the relative loss of thickness [(e₀−e(t))/e₀] as a function of the elongation (t) has a maximum value of between 5 and 50%, preferably less than or equal to 40%, even more preferably less than 35%, in particular less than 30%.

According to the invention, for a given initial thickness, the laminate, in the stretched state, is thicker than the laminates of the prior art, giving the user an impression of greater sturdiness when they stretch the laminate, in particular when it forms an elastic lug for closing the waist of a nappy. The laminate is also easier to manufacture and is softer to the touch and/or has a more bulky or voluminous visual appearance.

Preferably, the curve giving the relative loss of thickness [(e₀−e(t))/e₀] as a function of the elongation (t) has a maximum value of greater than 10%, even more preferably greater than 20%.

Preferably, the so-called maximum elongation according to the first direction or according to the second direction is between 50% and 100%.

Preferably, the ratio of the initial thickness (e₀) minus the thickness (eₘₐₓ) of the laminate at maximum elongation over the initial thickness (e₀), i.e. the ratio [(e₀−eₘₐₓ)/e₀], is less than or equal to 55%, in particular less than 40%, in particular less than 35%, and/or is greater than 5%, in particular greater than 10%, in particular greater than 20%.

Preferably, the ratio of the initial thickness (e₀) minus the thickness (e₁₀₀) of the laminate at 100% elongation over the initial thickness (e₀), i.e. the ratio [(e₀−e₁₀₀)/e₀], is less than or equal to 55%, in particular less than 40%, in particular less than 35%, and/or is greater than 5%, in particular greater than 10%, in particular greater than 20%.

Preferably, the laminate, after stretching to an elongation of 100% proceeding from the initial state before its first stretching, and then relaxation to the non-stretched state (0% elongation), has a thickness (eᵥ) greater than the initial thickness (e₀), and the ratio [(eᵥ−e₀)/e₀] is greater than 2%, in particular greater than 5%, more particularly greater than 10%, and is preferably less than 70%, in particular less than 50%, more particularly less than 35%.

Preferably, the laminate, after stretching to an elongation of 100% proceeding from the first non-stretched state and then relaxation to a second non-stretched state (0% elongation), has a thickness (eᵥ), and the ratio of the thickness (eᵥ) minus the thickness (e100) of the laminate at 100% elongation over the thickness, i.e. the ratio (eᵥ) [(eᵥ−e100)/eᵥ], is less than or equal to 60%, in particular less than 50%, in particular less than 40%, in particular less than 35%.

According to a preferred embodiment, fixing of the at least one film to the at least one nonwoven layer is performed by interposition of an adhesive, for example glue, along a strip or a plurality of strips or lines of glue extending lengthwise in the second direction, in particular MD, and at a distance from one another in the first direction, in particular CD, with a constant or variable pitch and/or discontinuities in the direction MD.

According to another embodiment, fixing of the at least one film to the at least one nonwoven layer is performed by hot lamination of the elastic film on at least one of the two nonwovens, in some cases by hot lamination of the elastic film on two upper and lower nonwovens.

According to yet another embodiment, fixing of the at least one film to the at least one nonwoven layer is performed by hot calendering and/or by ultrasonic welding.

Preferably, the at least one nonwoven layer comprises continuous filaments.

Preferably, the at least one nonwoven layer comprises crimped filaments.

Preferably, the at least one nonwoven layer comprises a spunbond nonwoven.

Preferably, the at least one nonwoven layer comprises a nonwoven obtained by melting, in particular a layer of spunbond (S), in particular a spunbond based on crimped filaments, and a layer of meltblown (M), or a combination of a plurality of these layers (for example SM, SMS, SMMS, SSMMS, SSSMMS, SSSMMSS).

According to another aspect of the invention, itself forming an invention independent of the aspect(s) above but which can advantageously be implemented in combination therewith, the laminate, after stretching for the first time to an elongation of 100%, and then relaxation to a second non-stretched state (0% elongation), has a thickness (eᵥ) greater than the initial thickness (e₀) before it is stretched for the first time, and the ratio [(eᵥ−e₀)/e₀], is greater than 2%, in particular greater than 5%, more particularly greater than 10%, in particular greater than 12.5%, even more preferably greater than 15%, and/or is preferably less than 70%, in particular less than 50%, more particularly less than 35%.

According to this aspect, it is thus possible to roll a greater length of a laminate that is not yet stretched (at the output of the production line) onto a reel of constant diameter, while having available a laminate of the same quality in use, or to roll a same length of laminate that is not yet stretched, onto a reel of constant diameter, while having available a laminate of better quality in use.

According to yet another aspect of the invention, itself forming an invention independent of the preceding aspects but which can advantageously be implemented in combination with each or a plurality thereof, an elastic laminate, extending widthwise in a direction CD and lengthwise in a direction MD, comprises:
at least one nonwoven layer; and
at least one elastic film fixed by an upper face or lower face, respectively, to a lower face or upper face, respectively, of the at least one nonwoven layer, is characterised in that

the curve giving, during the first stretching, the force (N) applied to the laminate in the direction CD as a function of the stretching (elongation or extension as a %) having an inflection point that separates a first lower curve segment having its concavity turned downwards, and a second upper curve segment having its concavity turned upwards, the first lower segment having a lower apex point and the second upper segment having an upper apex point, the portion of the curve between the origin and the lower apex point being the initiation segment of the curve, the portion of the curve between the lower apex point and the inflection point being the usage segment of the curve, and the portion of the curve between the inflection point and the upper apex point being the stop segment of the curve, and the X-axis of the initiation segment extends between 0% and a value of between 5% and 20%, for example 10%, and the X-axis of the usage segment extends between a value of between 5% and 20%, for example 10%, and a value of between 60% and 80%, for example 70%, while the X-axis of the stop segment extends, following the usage segment, up to a value of between 80% and 120%, in particular, as mentioned in the paragraph above, 100%.

According to a favourable embodiment, the laminate is such that, in a non-stretched state of the laminate and/or of the elastic film, the nonwoven layer comprises, alternately, in the transverse cross-section CD, intermediate sections, in particular straight or substantially straight, in particular curved, in particular having their concavity turned upwards, extending in the direction CD while being fixed to the elastic film, and inverted U-shaped curved sections, in particular in the shape of a Ω, each consisting of two strand sections proceeding from two successive intermediate sections and meeting at an apex, which are not fixed to the elastic film and remain at a distance therefrom, so as to define empty spaces between them and the elastic film, the curvature of the curved sections being greater than that of the intermediate sections.

By thus providing that the nonwoven layer is fixed to the elastic film only intermittently, a type of limitation of the possible stretching of the laminate is obtained, in particular when the temperature at which the laminate is used, for example when it is used on a nappy, increases significantly, for example approaching 30° C., which has the effect of significantly increasing the stretching capacity of the elastic film. Thus, in particular at this temperature, when the user stretches the laminate in the direction CD, at the moment when the inverted U-shaped sections flatten out between the two feet, in parallel with the direction of stretching and with the fixing section, they feel a type of stop, prompting them not to stretch the laminate any further, even though, at these body temperatures, they could stretch the elastic much further, to the point that the long-term stability of the laminate deteriorates.

The laminate is thus prevented from deteriorating too rapidly, by combatting the phenomenon of “neckdown” or shrinkage of the elastic which appears, for example in the case of a nappy, gradually during use of the nappy, for example by being opened and then closed, repeatedly, in particular when it is stretched too much. Furthermore, a laminate is obtained that is easier to manufacture and is softer and/or has a more bulky or voluminous visual appearance.

Preferably, the intermediate sections, in particular straight or substantially straight intermediate sections, in particular when they are curved with their concavity turned upwards, each extend over a distance, measured in the direction CD, which is less than 10% of the distance, measured in the direction CD, between the two intermediate sections from which the two strand sections of an inverted U-shaped section, in particular in the shape of a Ω, originate, in particular is between 10% and 50% of this distance.

According to yet another aspect of the invention, itself constituting an invention independent of the aspects above, but which can advantageously be implemented in combination with each or a plurality of said aspects, the elastic film based on an elastomer composition does not have an outer skin, the elastic film preferably being sufficiently adhesive and/or tacky to hold the nonwoven on one or more intermediate sections without glue.

According to yet another aspect of the invention, the elastic film comprises a skin, preferably two skins, forming an outer surface of the elastic film.

Very preferably, at least one strip or line of adhesive, in particular glue, is located between the elastic film and a respective intermediate section.

Preferably, the height of the inverted U, in particular in the shape of a Ω, (height of the apex point) is greater than its width (distance between the two feet of the strands of the inverted U.

In particular, in a favourable manner, each inverted U is in the shape of a Ω, and is defined by two left-hand and right-hand strands originating from a respective straight section and meeting at the apex of the inverted U, and one of the two left-hand or right-hand strands of at least one of the inverted U-shaped sections, in particular the two left-hand and right-hand strands of at least one inverted U-shaped section has/have, in a starting zone proceeding from the straight section from which it/they originate, a respective curved shape, the concavity of which is turned towards the elastic film.

Preferably, an additional nonwoven layer is provided, on the other side of the elastic film, said additional layer being a nonwoven layer based on short fibres consolidated by water jets or thermally, in particular a hydrobonded carded nonwoven (spunlace) or a carded thermobonded nonwoven.

Preferably, the nonwoven layer has a first basis weight and the additional nonwoven layer has a second basis weight which is different from the first basis weight, in particular greater than the first basis weight, in some cases the first and second basis weights being equal to within 20%.

In particular, the nonwoven layer has a first apparent basis weight, and the additional nonwoven layer has a second apparent basis weight, in the elastic zone, the first apparent basis weight is greater than the second apparent basis weight, the apparent basis weight being determined by measuring, in a cross-sectional view according to an axis CD, the evolution of the product and, by multiplying the evolution by the basis weight of the nonwoven in the non-stretched and/or non-shaped state.

In particular, the nonwoven layer has a first rigidity and the additional nonwoven layer has a second rigidity which is less than the first rigidity, in particular the nonwoven layer has an elongation in CD at 5 N of less than 70%, and the additional nonwoven layer has an elongation in CD at 5 N of greater than 70%.

According to one example, the length of the elastic film in the first direction, in particular CD, of the laminate is less than the width of the lap or nonwoven layer in the first direction, in particular CD, of the laminate.

According to one example, which may be combined or not with the previous example, the width of the elastic film in the second direction, in particular MD, of the laminate is equal or substantially equal to the length of the lap or nonwoven layer in the second direction, in particular MD, of the laminate.

The present invention also relates to a method for producing a laminate, in particular a laminate according to the invention.

According to the invention, the method comprises the steps in which an elastic film is taken; a nonwoven layer, in particular having continuous filaments, in particular a spunbond, in particular a “crimped” spunbond, is taken; the nonwoven layer is shaped to obtain a nonwoven layer in the shape of waves having, in transverse cross-section, the shape of a succession of inverted U shapes which are separated by intermediate sections, in particular straight or substantially straight, or curved or substantially curved, having their concavity turned upwards, the curvature of the curved sections being greater than that of the intermediate sections, the curvature of the latter being in particular zero when they are straight and close to zero when they are substantially straight; and the wave-shaped nonwoven layer is fixed to the elastic film, preferably in a non-stretched state thereof, by the side of the layer of the nonwoven that is opposite the crests of the waves.

Preferably, the filaments are oriented in the direction MD.

Preferably, in order to form (shape) the nonwoven layer, said layer is passed between the toothed or serrated rollers.

Preferably, prior to fixing the wave-shaped nonwoven layer to the elastic film, a strip of adhesive, in particular of glue, is deposited over the entire width of the elastic film, and the wave-shaped nonwoven layer is then laminated onto the elastic film.

According to another embodiment, the elastic film is hot-extruded between a support roller and the waver into which the upper nonwoven passes.

The present invention also relates to a nappy for a baby or adult incontinence pants comprising at least one laminate according to the invention, in particular in order to form the hook tabs originating laterally from the rear waist of the nappy or incontinence pants, such that the hooks engage with loops originating from the front face of the waist of the nappy, in order to achieve closure of the nappy or incontinence pants.

The present invention also relates to an elastic laminate with hooks, comprising a laminate according to the invention and at least one lap with hooks fixed to the laminate, in particular on the upper nonwoven layer, preferably in a zone without a wave or inverted U.

The present invention also relates to a roller comprising a laminate according to the invention, said laminate having a length at least greater than 1 meter and being rolled according to an axis perpendicular to the axis MD, said laminate comprising two adjacent elastic films.

The present invention also relates to a use of a laminate according to the first aspect of the invention, for obtaining an elastic laminate having an apparent capacity, in particular when it is in the form of an elastic lug of a waist of a nappy, for being stretched without the user having the impression that the laminate will break due to the stretching, in particular a laminate having an initial thickness (e₀) before its first stretching, and when the laminate is stretched according to the first or the second direction up to a so-called maximum elongation, at least equal to 50%, the curve giving the relative loss of thickness [(e₀−e(t))/e₀] as a function of the elongation (t) has a maximum value of between 5 and 50%, preferably less than or equal to 40%, even more preferably less than 35%, in particular less than 30%.

Preferably, the curve giving the relative loss of thickness [(e₀−e(t))/e₀] as a function of the elongation (t) has a maximum value of greater than 10%, even more preferably greater than 20%.

Preferably, the so-called maximum elongation according to the first direction or according to the second direction is between 50% and 100%.

Preferably, the ratio of the initial thickness (e₀) minus the thickness (eₘₐₓ) of the laminate at maximum elongation over the initial thickness (e₀), i.e. the ratio [(e₀−eₘₐₓ)/e₀], is less than or equal to 55%, in particular less than 40%, in particular less than 35%, and/or is greater than 5%, in particular greater than 10%, in particular greater than 20%.

Preferably, the ratio of the initial thickness (e₀) minus the thickness (e₁₀₀) of the laminate at 100% elongation over the initial thickness (e₀), i.e. the ratio [(e₀−e₁₀₀)/e₀], is less than or equal to 55%, in particular less than 40%, in particular less than 35%, and/or is greater than 5%, in particular greater than 10%, in particular greater than 20%.

Preferably, the laminate, after stretching to an elongation of 100% proceeding from the initial state before its first stretching, and then relaxation to the non-stretched state (0% elongation), has a thickness (eᵥ) greater than the initial thickness (e₀), and the ratio [(eᵥ−e₀)/e₀] is greater than 2%, in particular greater than 5%, more particularly greater than 10%, and is preferably less than 70%, in particular less than 50%, more particularly less than 35%.

Preferably, the laminate, after stretching to an elongation of 100% proceeding from the first non-stretched state and then relaxation to a second non-stretched state (0% elongation), has a thickness (eᵥ), and the ratio of the thickness (eᵥ) minus the thickness (e100) of the laminate at 100% elongation over the thickness, i.e. the ratio (eᵥ) [(eᵥ−e₁₀₀)/eᵥ], is less than or equal to 60%, in particular less than 50%, in particular less than 40%, in particular less than 35%.

In particular, the laminate according to the invention has a greater length than width, in particular in a ratio greater than 1.1, in particular greater than 1.25, in particular greater than 2, in particular greater than 50, more particularly greater than 100, and preferably less than 10,000, 5,000, 2,000 or 1,000. In particular, the laminate is rolled and/or unrolled, in particular in its lengthwise direction.

By way of example, a plurality of embodiments of the invention will now be described, with reference to the drawings, in which:

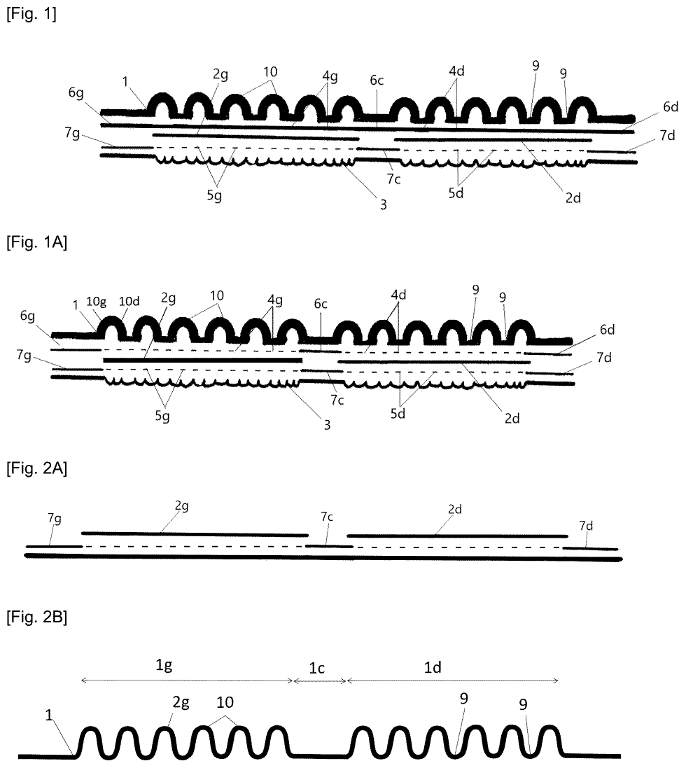

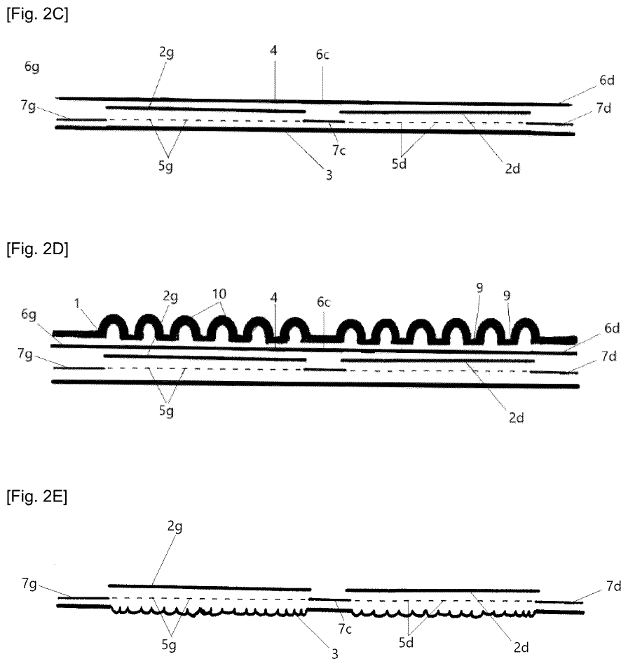

FIG. 1 is a schematic cross-sectional view, CD, of a preferred embodiment of a laminate according to the invention.

FIG. 1A is a schematic cross-sectional view, CD, of another embodiment of a laminate according to the invention.

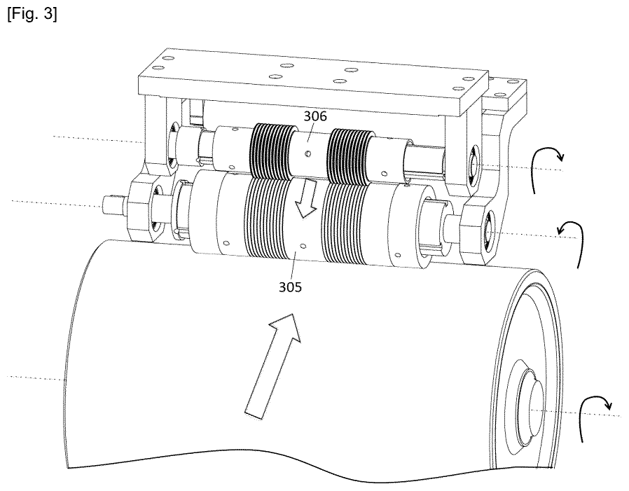

FIG. 2A-2E schematically show a plurality of steps during the production of the laminate in FIG. 1.

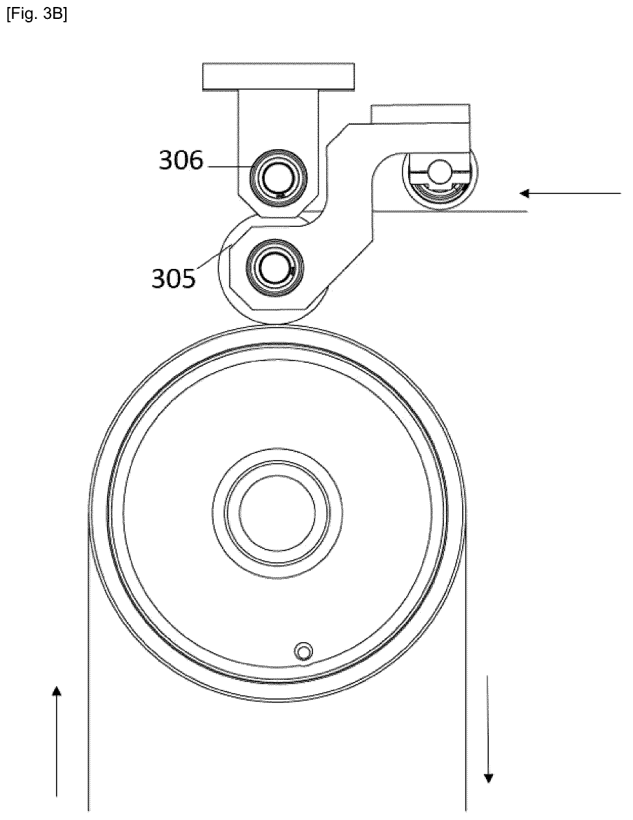

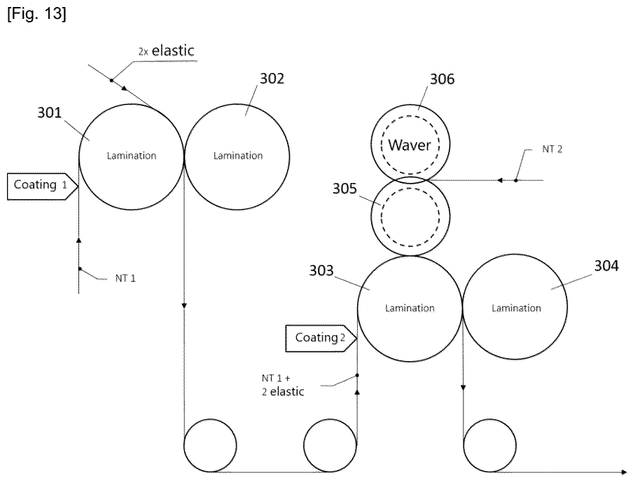

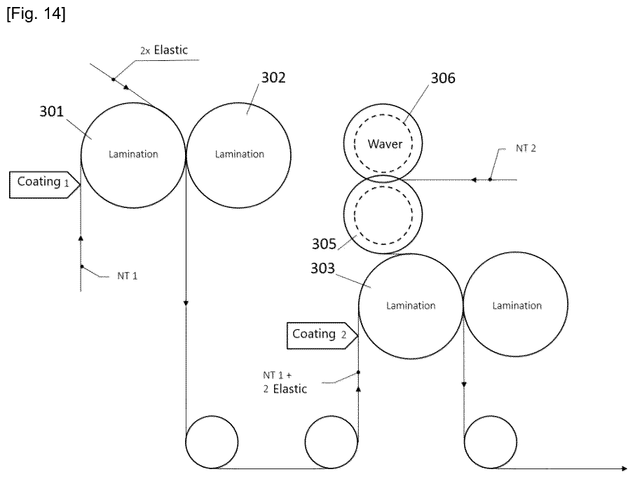

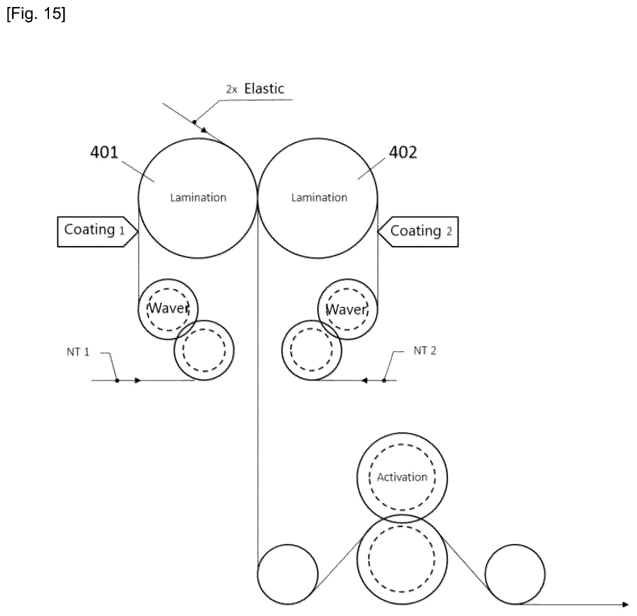

FIG. 3 is a schematic perspective view of one part of a “waver” device intended for forming/shaping the upper nonwoven by passing it between two toothed rollers, the teeth of which interpenetrate without contact in CD, said device forming part of the installations shown in FIGS. 13 to 16.

FIG. 3B is a side view of the device in FIG. 3.

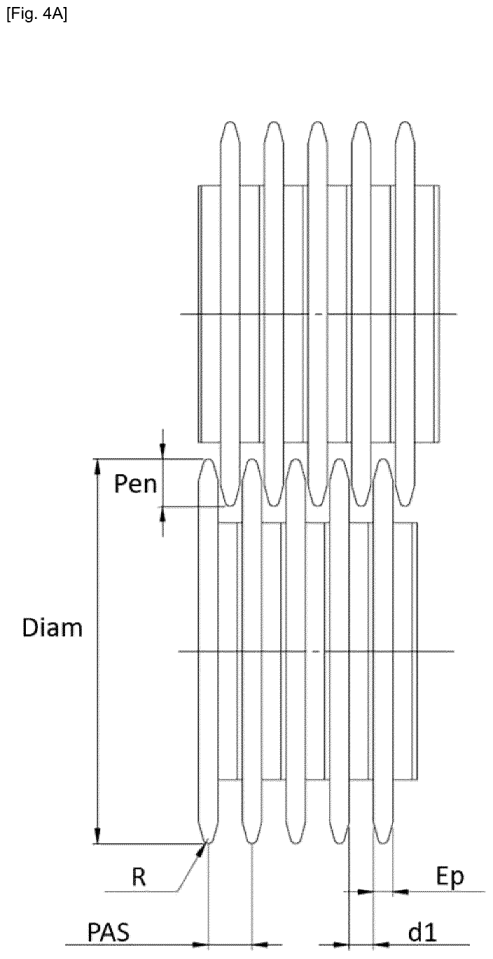

FIG. 4A shows a tooth profile of the two toothed rollers of the installation in FIG. 3, which can be used for shaping the upper nonwoven layer from FIG. 2B into waves.

FIG. 4B shows another possible tooth profile for the two toothed rollers of the installation in FIG. 3, which can be used for shaping the upper nonwoven layer from FIG. 2B into waves.

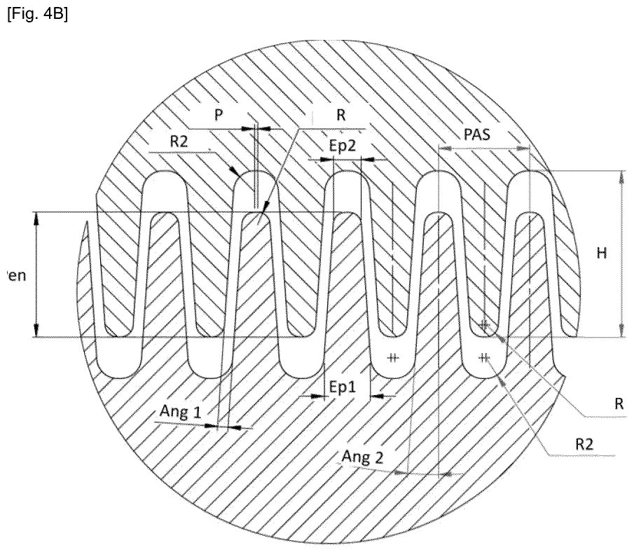

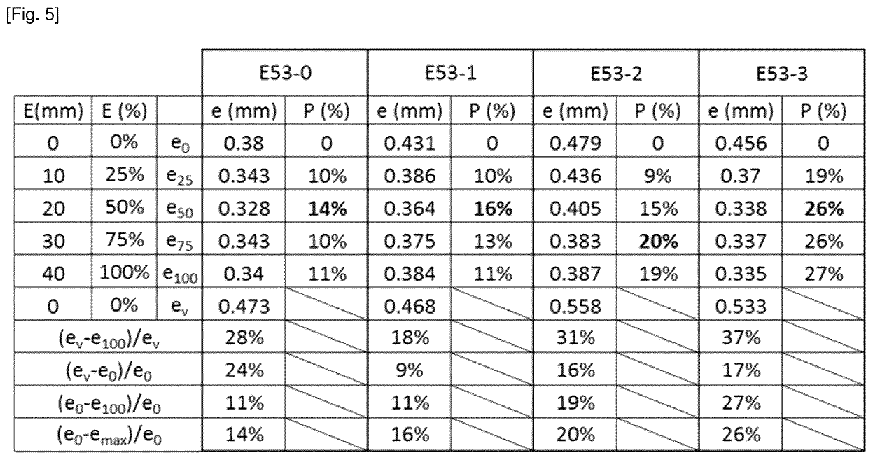

FIG. 5 is a table showing the measured thicknesses of four samples E53-0, E53-1, E53-2 and E53-3 depending on their stretching (the elongation being equal to the ratio of the increase in length over the initial length) for a first example of a laminate according to the invention.

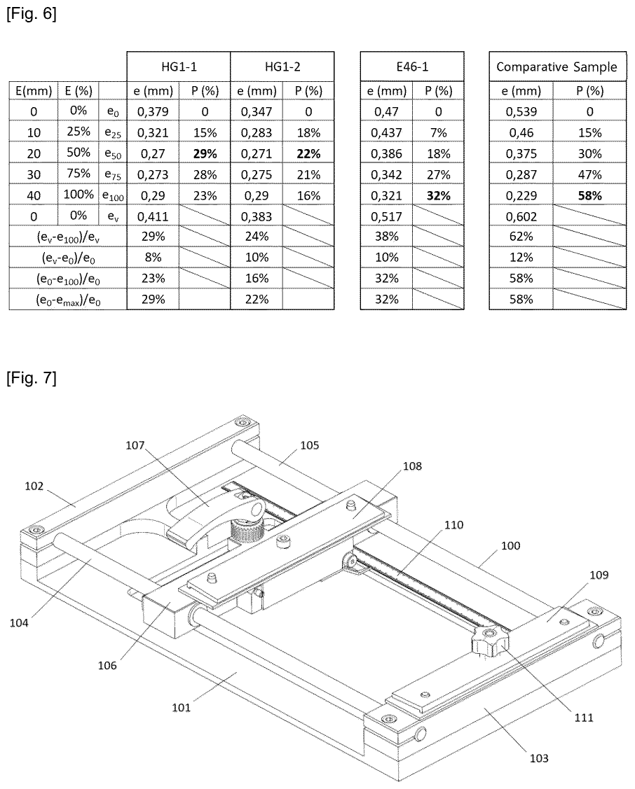

FIG. 6 illustrates four tables showing, as in FIG. 5, the measured thicknesses of three samples HG1-1, HG1-2 and E46-1 depending on their stretching (the elongation being equal to the ratio of the increase in length over the initial length) for the second, third and fourth examples of laminates according to the invention, as well as for a comparative (or reference) sample of a laminate of the prior art.

FIG. 7 is a perspective view of a tooling for support and adjustment of the stretching of a laminate, used for measuring the thickness thereof.

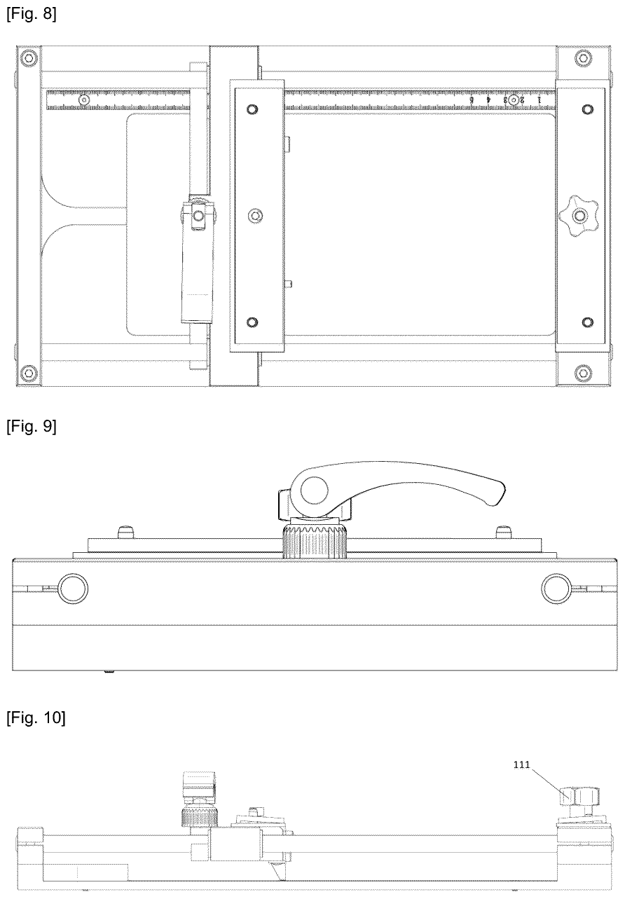

FIG. 8 is a plan view of the tooling in FIG. 7.

FIG. 9 is a side view of the tooling in FIGS. 7 and 8.

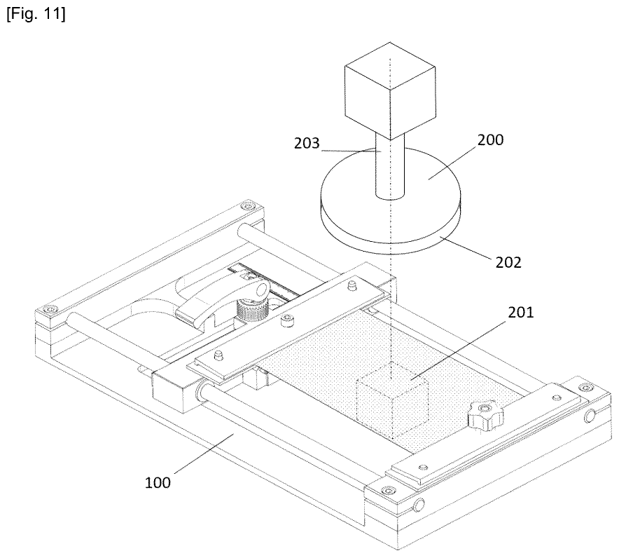

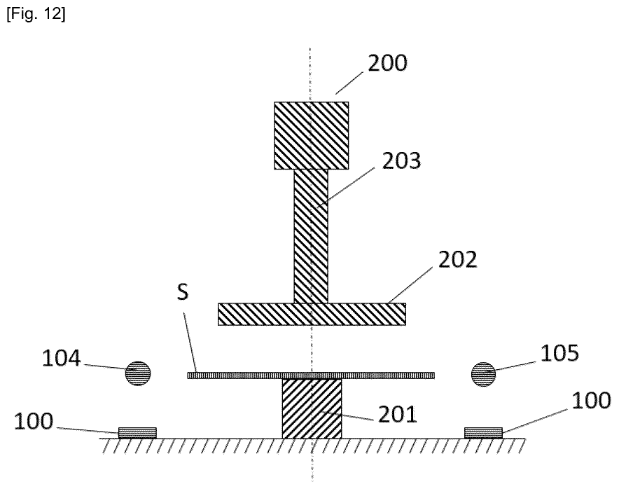

FIG. 10 is another side view of the tooling in FIGS. 7 to 9.

FIG. 11 is a perspective view of an installation for measuring the thickness of a laminate using the tooling for support and adjustment of the stretching shown in FIGS. 7 to 10.

FIG. 12 is a sectional view of the installation in FIG. 11.

FIG. 13 is a schematic representation of an embodiment of a production installation for a laminate of the invention.

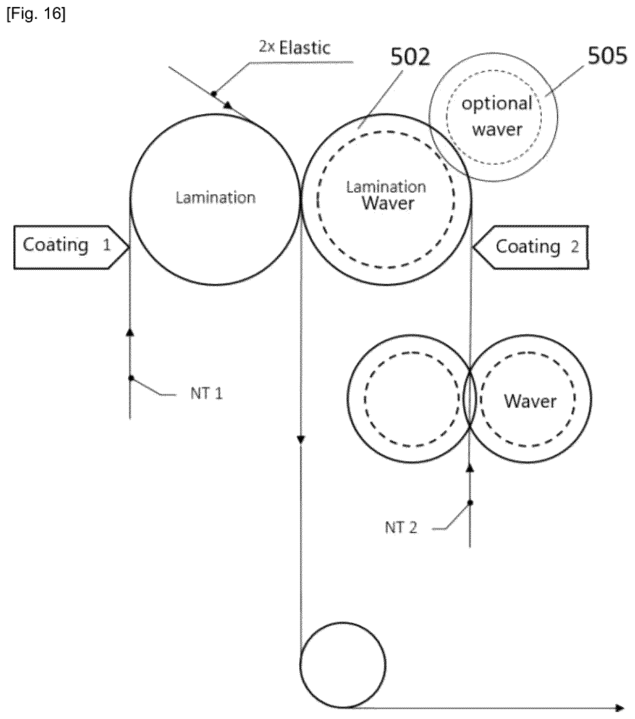

FIG. 14 is a schematic view of a variant of the production installation in FIG. 13.

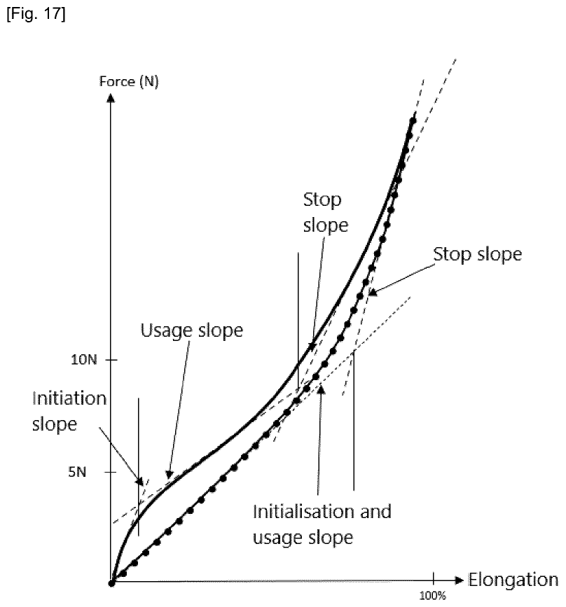

FIG. 15 is a schematic representation of another embodiment of a production installation for a laminate of the invention.

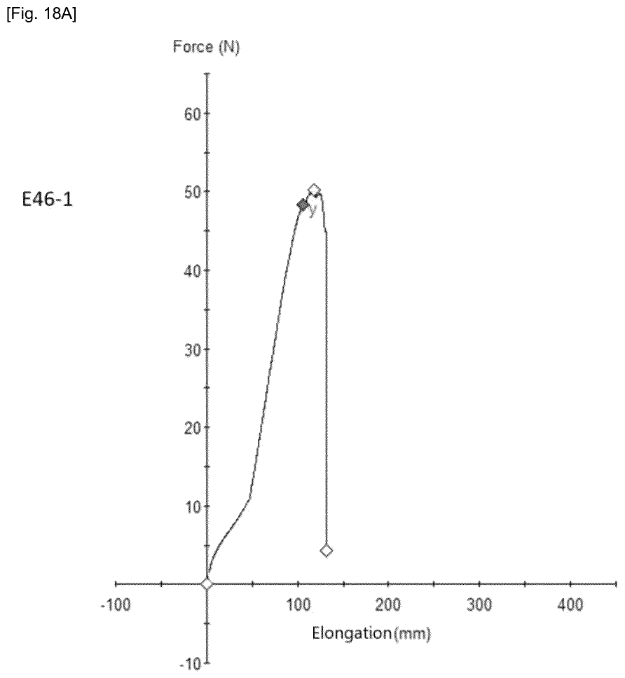

FIG. 16 is a schematic representation of yet another embodiment of a production installation for a laminate of the invention.

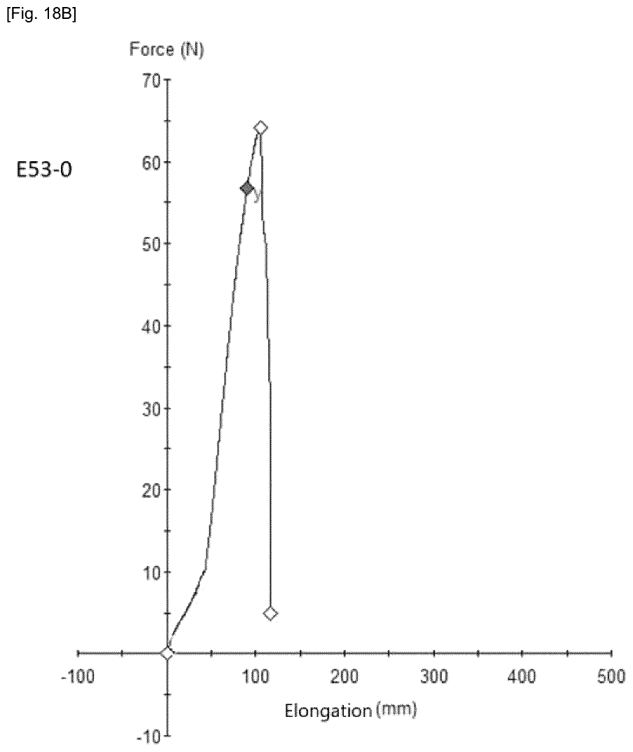

FIG. 17 shows two curves representing the force as a function of the extension (or elongation), one (solid line) for a laminate according to the invention making it possible to explain the concept of a stop and how to determine said stop for a laminate from its force curve (N) as a function of the extension (%), and the other (solid line with dots) for a laminate of the prior art.

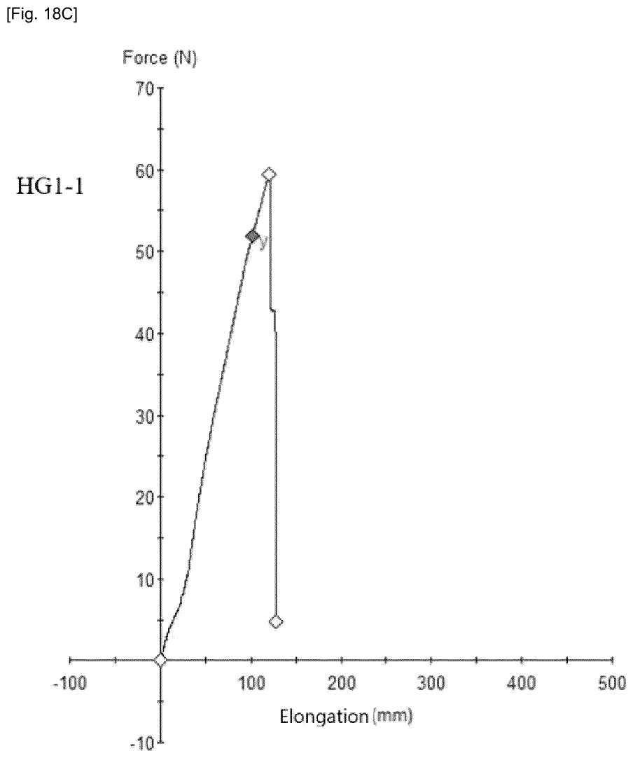

FIG. 18A-18C show the force/extension curves for laminates according to the embodiments E46-1, E53-0 and HG1-1 of the invention.

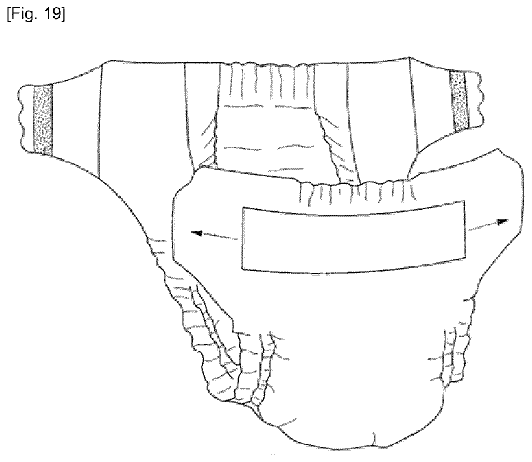

FIG. 19 is a perspective view of a nappy comprising two elastic lugs formed from a laminate according to the invention.

In FIG. 1, a laminate comprises, in succession from top to bottom, an upper nonwoven layer 1, two elastic films 2d, 2g, right-hand and left-hand, respectively, and a lower nonwoven layer 3. The upper nonwoven layer 1 comprises undulations.

The two elastic films 2d, 2g extend in the direction CD over a smaller distance than the distance over which the two nonwoven layers 1 and 3 extend, such that two edge regions and a central region without elastic film are formed on the left-hand and right-hand edges of the laminate and in the centre.

The upper nonwoven layer 1 and the elastic films 2d, 2g are fixed by interposition, therebetween, of a strip 4 of adhesive, in particular glue.

In the same way, the lower nonwoven layer 3 and the elastic films 2d, 2g are fixed by interposition, therebetween, of strips or lines 5d, 5g of adhesive, in particular glue, which extend lengthwise in the direction MD and are spaced apart from one another in the direction CD.

In the regions without elastics, the two nonwovens 1 and 3 are fixed directly to one another (i.e. without interposition of elastic film, only the adhesive being present therebetween) by fields 6d, 7d, 6c, 7c, 6g, 7g, respectively right, central and left, extending in the direction MD and having a width that is substantially equal to the respective width of the edge and central regions.

In cross-section CD, as in FIG. 1, the undulations of the upper nonwoven layer 1 form an alternating succession of intermediate sections 9 and sections 10 that are curved in an inverted U. The sections 9 extend in the direction CD over a respective width which may be identical for all the sections 9. However, they may have widths that vary from one section to another, without departing from the scope of protection of the invention. In the same way, the inverted U-shaped sections 10 may be identical to one another, having in particular a respective width (distance between the two successive straight sections to the left and to the right of the inverted U) which may be identical for all the inverted U-shaped sections 10, as well as a height of the U (maximum vertical distance between the apex and the elastic film) that is identical for all the inverted U shapes. However, they may have widths and heights that vary from one inverted U-shaped section to another, without departing from the scope of protection of the invention. The intermediate sections 9 may be straight or substantially straight, or curved, in particular slightly curved. Whether they be straight or substantially straight or slightly curved or curved, the sections 9 have a curvature which is always less than that of the inverted U-shaped curved sections. In the direction MD, the undulations extend continuously over the entire length of the laminate.

Each inverted U-shaped section comprises two sections in the form of a strand, respectively 10g left-hand and 10d right-hand, each originating from a respective intermediate section 9, which meet at the apex of the inverted U-shaped section.

Each of the two left-hand or right-hand strands of the inverted U-shaped sections has, in a starting zone proceeding from the straight section from which it originates, a respective curved shape, the concavity of which is turned towards the elastic film.

The inverted U-shapes may have a width (distance in parallel with the direction CD between the two straight sections from which the inverted U originates) of between 0.5 mm and 2 mm, and a height of between 0.5 mm and 2 mm, in particular between 0.9 mm and 1.8 mm.

The intermediate sections may have a length, in the direction MD, that is greater than 3 mm, in particular greater than 5 mm, in particular greater than 10 mm, and/or over the entire length in MD of the laminate and/or the entire length in MD of the laminate in the form of an elastic lug, in particular less than 100 mm. The intermediate sections extend in a continuous or discontinuous manner in the direction MD.

The intermediate sections may have a width of between 0.4 mm and 2 mm, in particular between 0.4 mm and 1.9 mm, in particular between 0.5 mm and 1.5 mm. In particular, said width is less than 10% of the width of the inverted U shapes, in particular is between 10% and 50% of said width, even more preferably between 10% and 30% of said width of the inverted U shapes in the non-stretched state.

As can be seen from FIGS. 1 and 1A, the two lower and upper nonwoven layers extend, in the direction CD, over the entire width of the laminate. Said width is the total width of the nonwoven. The two elastic films 2g, 2d each extend over a respective width that is less than half the width of the laminate. The sum of the two respective widths over which the two films extend represents the total elastic width. Here, the relation or ratio of the total elastic width to the total nonwoven width is between 0.4 and 0.8, and in particular is equal to approximately 0.7 in FIGS. 1 and 1A. FIGS. 1 and 1A show an embodiment in which the upper nonwoven 1 is shaped so as to have undulations which form, in cross-section, here CD, inverted U shapes that are separated by intermediate sections, for example straight sections, while in the lengthwise direction, here MD, these undulations extend without a break from one end of the laminate to the other. This shaping is performed in particular by means of a device referred to as a “waver” or “tamper” described below. In contrast, in this example, the lower nonwoven 3 has not been shaped by a “waver”, but only activated, as also explained below. It is possible, on the nonwoven shaped by the “waver”, to also perform a slight activation, if desired. Alternatively, the lower nonwoven 3 can also be formed identically to the upper nonwoven 1, and in particular be shaped by a “waver” or “tamper” such that it has, like the upper nonwoven 1, undulations which form, in cross-section CD, inverted U-shaped sections that are separated by intermediate sections, in particular straight, while in the direction MD the undulations extend continuously from one end of the laminate to the other.

The curved sections 10 of the upper nonwoven extend in the direction CD over a total curved section width, and the intermediate sections 9 extend over a total intermediate section width, the ratio equal to the total curved section width over the total width of the nonwoven being between 0.25 and 0.85, in particular between 0.35 and 0.75, and in particular being equal to approximately 0.45 in FIGS. 1 and 1A.

FIG. 1A shows another embodiment in which the continuous strip 4 of glue of the embodiment in FIG. 1 is replaced by strips or lines 4d, 4g which extend lengthwise in the direction MD and are spaced apart from one another in the direction CD. The strips or lines of glue each have a width in the direction CD which may be identical or may vary from one line/strip to the other.

The width of the lines or strips of adhesive, in particular glue, may be between 0.5 mm and 3 mm, or between 10 mm and 80 mm.

Furthermore, according to an advantageous embodiment, at least one of the lines or strips of glue is located between an elastic film 2d, 2g and one of the intermediate sections 9 of the upper nonwoven layer 1, and has a width in the direction CD that is strictly less than that of said one of the straight sections 9. In particular, some of the lines or strips of glue, in particular all the lines or strips of glue, are each located between an elastic film 2d, 2g and a respective one of the intermediate sections 9 of the upper nonwoven layer 1, and each have a width in the direction CD that is strictly less than that of said one respective one of the intermediate sections 9.

According to another different embodiment, all the lines or strips of glue are each located between an elastic film 2d, 2g and a respective one of the sections 9 of the upper nonwoven layer 1, and each have a width in the direction CD that is equal to that of said one respective one of the sections 9.

The upper nonwoven layer 1 is formed from a nonwoven based on continuous filaments, i.e. filaments with a long length, in particular 120 mm or more. In particular, the nonwoven of the nonwoven layer 1 may be a spunbond or comprise a spunbond associated with other nonwovens, for example an SM, an SMS, or the like.

The upper nonwoven layer 1 may have a basis weight of between 10 and 30 g/m², in particular between 10 and 22 g/m².

The lower nonwoven layer 3 may also be formed from continuous filaments, but, and preferably, also from any other nonwoven, in particular based on short fibres, in particular a carded material, such as a spunlace.

The lower nonwoven layer 3 may have a basis weight of between 10 and 30 g/m², in particular between 18 and 25 g/m².

The thickness of the upper nonwoven layer (measured perpendicularly to the straight sections, in the resting state and in fresh air without application of pressure on the nonwoven) may be between 0.5 mm and 2 mm.

The thickness of the lower nonwoven layer (measured perpendicularly to the straight sections, in the resting state and in fresh air without application of pressure on the nonwoven) may be between 0.1 mm and 0.5 mm.

FIG. 2A schematically shows an intermediate laminate obtained during a step during manufacture of the laminate in FIG. 1 in an installation such as that shown in FIG. 13 or 14.

The strips of glue 5d, 5g, as well as the fields of glue 7d, 7c and 7g, have been deposited on a nonwoven layer, for example spunlace, 25 g/m², intended for forming the lower nonwoven layer 3, and the two elastic films 2g, 2d have been deposited on top, in order to obtain the intermediate laminate in FIG. 2A.

Alternatively, a precursor nonwoven layer intended to form the upper nonwoven layer has been shaped by passing it into the installation shown in FIGS. 3 and 4 between two rollers 305, 306 each comprising teeth which interpenetrate without contact in CD in order to shape the nonwoven layer, in the zones which have passed between the teeth, into an accordion shape, i.e. the two accordion zones 1d and 1g separated by a non-shaped central zone 1c (which has not passed between the teeth). Furthermore, two edge zones extend between the respective edges of the nonwoven layer and the two accordion zones, said two edge zones likewise not having passed through the teeth and not having been shaped. During passage into the installation having toothed rollers forming a “waver” module, the upper nonwoven layer that is shaped as shown in FIG. 2B is obtained. The waver module, unlike an activation module that is well known in the field for activating a laminate of this type to release the elasticity of the elastic film in the laminate, shapes the product into the form of a wave without breaking it, in particular without breaking the nonwoven. The waver shapes the product in such a way that the width of the nonwoven at the entrance to the waver is wider than the width of the laminate at the exit from the waver, in contrast to activation which aims to obtain an identical or larger width at the exit from the activation block. The nonwoven, which has been shaped or “waved” in at least one “waved” zone, has a quantity or mass of fibres per unit of width of the laminate which is greater, in the regions that have undergone the shaping, in particular in line with or facing the two elastic films, than the quantity or mass of fibres per unit of width of the laminate in the regions that have not undergone the shaping, for example in FIGS. 1 and 1A in the two lateral end regions without elastic film and in the central region also without elastic film. At the same time, in these regions that have undergone the shaping, the fibres and/or the filaments that make up the nonwoven have an average diameter that is substantially identical to the average diameter of the fibres and/or filaments in the regions that have not undergone the shaping. In particular, the relative difference between the two average diameters in a shaped zone and in a non-shaped zone is less than 15%, particularly less than 10%, in particular less than 5%, these variations corresponding to the typical variations in diameters that are encountered for fibres and/or filaments of nonwovens. It is possible to recognise a “waved” zone of nonwoven by observing the fibres and/or filaments of the zone, which, unlike a zone that has undergone an activation operation, does not have any broken fibres and/or filaments, and/or local microzones without fibres and/or filaments, and/or microzones having significant variation in the diameters of the fibres and/or filaments. The features of the microzones must be observed outside the points of consolidation of the nonwoven, and/or outside the points of connection of the nonwoven to itself and/or to the elastic film and/or to another nonwoven, said connection points being formed by application of pressure and/or temperature with a view to deforming the fibres and/or filaments. Here, diameter of fibres and/or of filaments means the circle of maximum dimension that includes the cross-section of the fibre and/or of the filament and which can have a circular or substantially circular, oval or substantially oval, or a differently shaped, cross-section. The nonwoven layer shaped by “waver” has, in the non-stretched state of the laminate, in cross-section according to the first direction, in particular CD, of the laminate, a neutral fibre, the developed length of which is greater, particularly at least 10% greater, in particular at least 15% greater, more particularly at least 20% greater, than the width of the laminate, while in cross-section according to the second direction, in particular MD, of the laminate, it has a neutral fibre, the developed length of which is equal or substantially equal to the length of the laminate.

Subsequently, the strip 4 of glue as well as the fields of glue 6d, 6c and 6g are deposited on the intermediate laminate in FIG. 2A in order to obtain the intermediate laminate in FIG. 2C.

Subsequently, the nonwoven layer in FIG. 2B is deposited by lamination on the intermediate laminate in FIG. 2, in the non-stretched state, in order to obtain the laminate in FIG. 1. The intermediate laminate in FIG. 2C, prior to lamination with the nonwoven layer in FIG. 2B, may be activated or not, and/or the nonwoven of the intermediate laminate in FIG. 2C may be activated in advance.

FIG. 2E is an illustration of the laminate in FIG. 2A which has furthermore undergone an activation step, prior to the step of coating with glue and the step of lamination of the shaped nonwoven.

It is then possible to perform activation of the laminate in FIG. 2D in order to release and/or adjust the elasticity of the lower nonwoven layer and/or of the elastic film, by stretching it in the direction CD, which has the effect of “breaking” the fibres and/or filaments of the nonwoven or causing them to lose their cohesion in order to thereby enlarge said nonwoven. Following activation, the nonwoven retracts under the effect of the elastic films, in order to obtain the laminate in FIG. 1. Advantageously, the nonwoven in FIG. 2B can be activated prior to the step of shaping into waves.

According to a variant, it is also possible to perform activation of the lower nonwoven layer after formation of the laminate in FIG. 2A, in order to obtain the intermediate laminate in FIG. 2D on which the steps of placement of glue and then lamination of the nonwoven from FIG. 2B, as described in FIGS. 2C and 2D, are performed.

According to the invention, it is possible to use a glue such as glues of the non-reactive PSA (pressure-sensitive adhesive) type, for example H2465 by Bostick, or a reactive PU glue, in particular XPU18314 by Bostik. Preferably, these glues will have a chemical nature similar to the elastomer film. For example, if one of these glues is analysed using an infrared spectrometer, in order to identify the chemical functions, or using liquid chromatography to separate and quantify the substances, preferably traces of one or more components or their derivatives, of the material or materials of the elastomer film, will be identified.

Preferably, these glues are based on SIS, SBS, SEBS and SEPS, allowing for good affinity with the film due to the similar chemical materials.

Preferably, the glue layer has a basis weight of less than 15 g/m², in particular less than 12 g/m², more preferably less than 8 g/m².

In the present invention, a nonwoven is intended to mean a product obtained following formation of a lap of fibres and/or filaments which has been consolidated. The consolidation may be mechanical, chemical or thermal, and results in the presence of bonds between the fibres and/or the filaments. This consolidation may be direct, i.e. performed directly between the fibres and/or the filaments by means of welding, or it may be indirect, i.e. by means of an intermediate layer between the fibres and/or the filaments, for example a layer of glue or a layer of binder. The term “nonwoven” relates to a structure in the form of a tape or lap of fibres and/or filaments which are interlaced in a non-uniform or irregular manner, or at random. A nonwoven may have a single-layer structure or a structure comprising a plurality of layers. A nonwoven may be made from different synthetic and/or natural materials. The natural materials, by way of example, are cellulose fibres such as cotton, jute, paper pulp, linen or the like, and may also include recycled cellulose fibres, such as rayon or viscose (cellulose acetate). The natural fibres for a nonwoven material may be prepared by using various processes, such as carding. Synthetic materials, by way of example but without restriction thereto, include synthetic thermoplastic polymers, which are known to form fibres and/or filaments which include, without restriction thereto, polyolefins, for example polyethylene, polypropylene, polybutylene, and the like; polyamide, for example polyamide 6, polyamide 6.6, polyamide 10, polyamide 11, polyamide 12, and the like; polyesters, for example polyethylene terephthalates, polybutylene terephthalates, polylactic acids (PLA), and the like, polycarbonates, polystyrenes, thermoplastic elastomers, vinyl polymers, polyurethanes, and mixtures and co-polymers thereof. Some of these materials may be bioplastic, for example bio-sourced (for example bio-PE, PLA or PHA (polyhydroxyalkanoates), polyamide 11, viscose (cellulose acetate), and the like) and/or biodegradable (PLA and the like). In a general manner, fibres and filaments differ mainly in their length and their manufacturing method.

“Continuous filaments” means the individual elements, which are very long compared to the diameter of their cross-section, extruded in a continuous manner in order to directly form a nonwoven lap, which can then be consolidated by thermobonding or any other means making it possible to achieve the desired performance, and/or transport thereof. Preferably, the continuous filaments have a length greater than 120 mm.

“Fibre” is understood as the generic term for denoting a textile material or a textile material element of reduced length, less than the length of the continuous filaments, and able to be spun and/or used in the formation of nonwovens. Two types of fibres are distinguished, short fibres formed of discontinuous material of a short length less than 50 mm (preferably from 25 mm to 50 mm), and long fibres formed in a discontinuous manner and of a long length greater than 50 mm (preferably from 60 mm to 120 mm).

Unlike continuous filaments which are consolidated directly after extrusion, the fibres are usually oriented and organised in a lap during a carding step that is well known to a person skilled in the art. This lap can then be consolidated by thermobonding or any other means making it possible to achieve the desired performance, and/or transport thereof.

According to the present invention, “film” means a material of a sheet or wrap type, the length and the width of which are each much greater than the thickness (for example in a ratio of 10×, 50×, or even 1000× or more). Typically, a film has a thickness of less than 0.7 mm, in particular less than 0.5 mm or even thinner. In particular, a string or a thread or a set of strings and/or threads are not films.

By way of non-limiting examples of an elastomer material, the following can be cited: styrene/isoprene (SI), styrene/isoprene/styrene (SIS), styrene/butadiene/styrene (SBS), styrene-ethylene/butylene-styrene (SEBS), styrene-ethylene/propylene-styrene (SEPS) copolymers, or SIBS. Mixtures of these elastomers with one another or with non-elastomers which modify some characteristics other than elasticity may also be considered. For example up to 50% by mass, but preferably less than 30% by mass, of polymer may be added in order to modify some characteristics of the base materials (elasticity, heat resistance, processability, UV resistance, colouring, etc.), such as polystyrenes or poly a-methyl-styrene, epoxy polyesters, polyolefins, for example polyethylenes or some ethylene/vinyl acetates, preferably those having higher molar masses.

The elastomer material may in particular be a styrene-isoprene-styrene, available for example from the Kraton Polymers company, under the name KRATON D (registered trademark), or from the DEXCO POLYMERS LP company (United States) or TSRC (Europe) under the name VECTOR SBC 4211 (registered trademark), and/or under the name VECTOR SBC 4111 (registered trademark), and/or under the name VECTOR SBC 4411 (registered trademark), or a mixture of at least two of these materials. It is also possible to use TPE (thermoplastic elastomer) materials, in particular a thermoplastic elastomer of polyurethane, in particular ESTANE (registered trademark) 2102-75A-TPU from the LUBRIZOL company. It is also possible to use a styrene-butadiene-styrene, in particular VECTOR SBC 4461 (registered trademark) from the Dexco Polymers company a TSRC COMPANY LP. It is also possible to use a styrene-ethylene/butylene, or a styrene-ethylene-butylene-styrene (SEBS) copolymer sequence.

Without being exhaustive, the list can be supplemented by the use of all hydrogenated polyisoprene polymers such as styrene-ethylene-propylene-styrene (SEPS), styrene-ethylene-propylene-styrene-ethylene-ethylene-propylene (SEPSEP), hydrogenated polybutadiene polymers such as styrene-ethylene-butylene-styrene (SEBS), styrene-ethylene-butylene-styrene-ethylene-ethylene-butylene-ethylene-butylene-butylene (SEBSEB), styrene-butadiene-styrene (SBS), styrene-isoprene-styrene (SIS), styrene-isoprene-butadiene-styrene (SIBS), hydrogenated polyisoprene/butadiene polymer such as styrene-ethylene-ethylene-ethylene-propylene-styrene (SEEPS), and hydrogenated vinyl-polyisoprene/hydrogenated polyisoprene/polyisoprene/polystyrene triblock polymers, such as HYBRAR 7311, which are commercially available (Kuraray America, Inc., Houston, Tex.), and combinations thereof.

Block polymer configurations such as diblock, triblock, multiblock, multiblock, star and radial are also envisaged in this disclosure. In some cases, sequenced copolymers of or higher molar masses may be desirable. Sequenced copolymers are available from Kraton Polymers U.S. LLC of Houston, Tex. under the names, for example, Kraton D1184, Kraton FG1901 and Kraton FG1924, and under the names Septon 8007 by the Kuraray company. Dynasol is another possible supplier of these polymers.

It is also possible to use an isooctyl acrylate and acrylic acid copolymer, according to monomer ratios of 90/10, which is a thermoplastic having physical cross-linking in the absence of a cross-linking agent.

Other possible materials are polyolefin polymers, mainly ethylene and/or propylene copolymers, having elastomer characteristics, in particular originating from metallocene catalysis, such as VISTAMAXX VM-1120 (registered trademark), available from the Exxon Mobil Chemical company, or indeed rubber-loaded polymers.

Examples of thermoplastic elastomers based on polyolefins, which can be used in the elastomer film layers, include, inter alia, a crystalline polyolefin, for example a homopolymer or a copolymer of an alpha-olefin having 1 to 20 carbon atoms, and comprising 1 to 12 carbon atoms.

The homopolymers and the copolymers described below are examples of crystalline polyolefins.
(1) Ethylene homopolymer. Ethylene homopolymer may be prepared by means of any of the low-pressure and high-pressure processes.
(2) Ethylene copolymers, and no more than 10 mol % alpha-olefins other than ethylene, or vinyl monomers; for example, ethylene octene copolymer, available under the trademarks Engage 8407 or Engage 8842 (Dow Chemical, Houston, Tex.).
(3) Polypropylene copolymers; examples include polypropylene impact copolymer PP7035E4 and polypropylene random copolymer PP9574E6 (Exxon Mobil, Houston, Tex., or indeed polypropylene homopolymers).
(4) Polypropylene random copolymers, and no more than 10 mol % alpha-olefins-olefins other than propylene.
(5) Sequenced polypropylene copolymers, and no more than 30 mol % alpha-olefins other than propylene.
(6) Butene-1-butene homopolymer.
(7) 1-butene random copolymers, and no more than 10 mol % alpha-olefins-olefins other than 1-butene.
(8) 4-methyl-1-pentene homopolymer, 4-methyl-1-pentene homopolymer.
(9) 4-methyl-1-pentene random copolymers, and no more than 20 mol % alpha-olefins other than 4-methyl-1-pentene.

Alpha-olefins include, for example, ethylene, propylene, butene-1, 4-methyl-1pentene, 1-hexene, and 1-octene.

The polyolefin-based thermoplastic elastomers that are commercially available and intended to be used in the elastomer film layers include VISTAMAXX™ (propylene and/or ethylene-based elastomer, available from ExxonMobil Chemical, Houston, Tex.), INFUSE™ (olefin block copolymers, available from Dow Chemical Company, Midland, Michigan), VERSIFY™ (propylene/ethylene elastomer, in particular obtained via the technology “INSITE technology”) such as VERSIFY™ 4200 (Dow Chemical Company, Midland, Michigan), ENGAGE™ (ethylene octane copolymer, available from Dow Chemical, Houston, Tex.) and NOTIO 0040 and NOTIO 3560 (available from Mitsui Chemical (USA), New York, N.Y.), Adflex X100 G (available from Lyondellbasell). The following materials could also be used:
a propylene elastomer, in particular the “Vistamaxx” range by EXXON MOBIL under the references 6000 and/or 6102 and/or 6102FL and/or 6202 and/or 6202FL and/or 6502, 7050BF and/or 7810, and/or
an olefin block copolymer, in particular the “Infuse” range by DOW CHEMICAL under the references 9000 and/or 9007 and/or 9010 and/or 9077 and/or 9100 and/or 9107, and/or
a polyolefin elastomer, in particular the “Engage” range by DOW CHEMICAL under the references 8402 and/or 8401 and/or 8411 and/or 8407 and/or 8137 and/or 8200 and/or 8207, and/or
a propylene-ethylene copolymer, in particular the “Versify” range by DOW CHEMICAL under the references 3200 and/or 3300 and/or 3401 and/or 4200.

In a particularly suitable embodiment, the polyolefin-based thermoplastic elastomer is VISTAMAXX™ 6102FL or VISTAMAXX 7050 BF (available from ExxonMobil Chemical, Houston, Tex.). The reference “™” for registered trademarks corresponds to “Trade Mark”.

In another case, the thermoplastic elastomer may be a thermoplastic ester/ether elastomer.

“Elastomer material” means a material which can be stretched without breaking under the effect of a stretching force exerted according to a given direction, and which can substantially return to its initial shape and dimensions after this stretching force is relaxed. This is for example a film which retains a residual deformation or set following elongation and relaxation (residual deformation also referred to as “permanent set” or “SET”) that is less than or equal to 30%, preferably less than or equal to 20%, yet more preferably less than or equal to 10%, of the initial dimension thereof (prior to elongation) for an extension of 100% of the initial dimension thereof, at ambient temperature (23° C.—degrees Celsius). The elastomer material may be a thermoplastic elastomer material, in particular a physically cross-linked thermoplastic elastomer material, such as those described in the present disclosure, or a chemically cross-linked thermoplastic elastomer material.

In order to measure the rigidity of a nonwoven layer, it is possible in particular to use the following test:

The rigidity can be measured using a constant elongation rate of a traction rack under elongation, for example a constant rate of 508 mm/min, comprising a computer interface (an appropriate instrument is an MTS Alliance with TestWorks 4 software, available from the MTS Systems Corp company, Eden Prairie, Minnesota), equipped with a 5 N or 10 N or 100 N load cell. The tests are performed at 23° C.+−2° C. and at approximately 50% to +−2% relative humidity.

The rack comprises two lateral clamping jaws, for example made of stainless steel, defining therebetween a gap, and a plunge blade, made of a “lightweight” metal such as aluminium, which is arranged centrally, halfway between the jaws, above the gap. The sample is positioned such that two end parts of the region of a nonwoven layer, in which it is desired to study the rigidity of one point in the centre, are each clamped in one of the jaws, such that the point of the sample at which it is desired to measure the rigidity is located exactly below the plunge blade.

Samples measuring 50 mm in width and more than 40 mm in length are cut, in order to allow for testing of an inter-jaw length of 40 mm. If the element does not have sufficient material for samples of this size, the available dimensions are used to compare the rigidity of samples of the same dimensions. The plunge blade is lowered at a constant speed of 508 mm/min over a vertical distance of 40 mm, regulating the acquisition frequency to 100 Hz.

The software is programmed to calculate the maximum peak bending force, and the rigidity (N/m) of the formed curve giving the force (N) as a function of the elongation (m). The rigidity is calculated as being the gradient of the curve due to bending/elongation for the linear region of the curve, using a minimum line segment of at least 25% of the total peak bending force to calculate the gradient.

FIGS. 7 to 10 show an example of tooling 100 for support and adjustment of a sample of a laminate with the aim of determining its thickness.

The tooling comprises a rectangular base 101 from which two low walls 102, 103 project laterally to the left and right, between which two front and rear rails 104, 105 extend in the form of rods. A trolley 106 is mounted such that it can slide along the two rails 104 and 105. Clamping means are provided, making it possible to lock the trolley 106 in a position of choice along the rails 104, 105. These means are controlled by a handle 107 which can be pivoted manually between a locking position in which the trolley 106 can no longer slide along the rails, and an unlocked position in which the trolley 106 can slide along the rails.

A movable jaw 108 is mounted on the top of the trolley 106, and a fixed jaw 109 is mounted on the top of the right-hand lateral low wall 103, the clamping of said jaws being able to be controlled by a clamping nut 111.

In order to support a rectangular laminate sample, for example of 30 mm by 30 mm, it is ensured that its two opposite edges in the direction CD are each held in one of the two movable 108 and fixed 109 jaws. Subsequently, the stretching (extension) which is intended to be applied to the sample is adjusted by releasing the handle 107 in order to thus cause the movable jaw 108 to slide. A slide 110 with markers makes it possible to know the value of the extension depending on the position of the movable jaw.

In order to measure the thickness of the sample of laminate S that is supported and stretched by the tooling 100 or an equivalent tooling, it is possible to use a pressure application and thickness measuring apparatus 200, as shown in FIGS. 11 and 12, in particular the apparatus known as “Precision Thickness Tester” marketed by the VVC company under reference D-2005-V.

Said apparatus 200 comprises a disc 202, having a circular surface with a surface area of 25 cm², arranged at the end of a cylindrical arm 203 that is movable vertically under the control of electronic control means integrated in the apparatus. The other end of the arm has an additional mass for applying a pressure of 1 KPa to a surface area of 25 cm². In order to perform these measurements, a 15 mm×15 mm square pallet 201 is used, and thus the force applied to the sample is 11.1 kPa (11.1 kPa=1 kPa*(25 cm²/2.25 cm²)) and not 1 kPa. Thus, once the laminate is clamped between the pallet and the disc, it is possible to measure the thickness of the sample in the region of the surface of the pallet, by performing the following steps:
the “zero” button of the apparatus is pressed, in order to move the arm downwards until it comes into contact directly (without a sample) on the 15×15 mm pallet, and to define the value of 0 micrometres,
the thickness is measured in order to check that the value of 0 micrometres is obtained, then
the thickness is measured with a larger weight, for example 10%, in order to check that the value of 0 micrometres is still obtained.

The sample is then positioned on the top of the 15×15 mm (2.25 cm²) pallet (i.e. 11.1 kPa=1 kPa*(25 cm²/2.25 cm²)), the 15×15 mm pallet being positioned in the axis of the cylindrical arm, and
the measurements are performed according to the different amounts of stretching adjusted for example by means of the support and adjustment tooling 100 shown in FIG. 11, or an equivalent tooling.

In order to obtain the curve showing the relative loss of thickness as a function of elongation, the laminate is stretched to at least four stretching values, for example 25%, 50%, 75% and 100%, and, for each stretching value, the thickness, for example e₂₅, e₅₀, e₇₅ et e₁₀₀, is measured according to the method set out above, and the straight lines connecting the successive points, thus determined, are plotted, in order to obtain said curve.

FIGS. 5 and 6 show the thickness measurement data as a function of stretching samples of four examples of laminates according to the invention and one comparative example of a laminate of the prior art.

The laminate of the prior art is an elastic laminate taken from a nappy having reference “02184499341631” from the pack of nappies with the Pampers® Baby-Dry™ trademark in size 4, and having reference “02184499 34 16:13 May 8, 2020 MADE in Germany E”. This product comprises an elastic film sandwiched between two nonwovens, said elastic film being fixed, by ultrasonic welding, in the stretched state to the two nonwovens.

In the present invention, maximum elongation of a laminate means the elongation in the direction CD, from which at least one undulation appears, in a cross-section of the laminate in the direction MD. In particular, in the present invention, the test is not carried out beyond 100% extension. The maximum loss of thickness is taken at 100%. The maximum extension is between 50% and 100%. If necessary, the maximum loss of thickness is taken at an elongation of 100%.

FIGS. 13 to 16 are schematic drawings showing production installations that make it possible to obtain laminates, in particular of the invention.

In FIG. 13, two first lamination rollers 301 and 302 define a lamination gap, between which a first nonwoven layer NT1 is passed, on which, at a first coating point, Coating 1, lines of glue have been deposited by means of a foil. Two elastic films are also passed into the gap side-by-side and at a distance from one another, in order to thus bring about, in the lamination gap, their fixing by lamination to the first nonwoven layer.

The intermediate laminate, formed of the first nonwoven layer NT1 and the two elastic films, is then transported towards a second lamination gap formed between two second lamination rollers 303, 304.

A second coating point, Coating 2, is arranged upstream of the second lamination gap and ensures the deposition on said intermediate laminate, from the die of the elastic films, of lines of glue, by means of a foil.

In addition, a second nonwoven layer NT2 passes into the second gap. Upstream of the second gap, the second nonwoven layer passes into a wave-formation gap, known as a waver, formed of two toothed rollers 305, 306, referred to as waver rollers, the teeth of which interpenetrate without contact in CD as shown in FIGS. 3 and 4B, which go on to shape the second nonwoven layer into the shape of waves, before it is introduced into the second gap in order to be laminated to the intermediate laminate, in order to thus obtain the final laminate at the exit from the second gap.

In the embodiment in FIG. 13, the second nonwoven layer, at the exit from the wave formation gap, is applied to the intermediate laminate before they are introduced into the second lamination gap, the support roller of the two waver rollers being in contact with one of the two second lamination rollers 303, 304.

The relative positions of the two waver rollers makes it possible to adjust the shape of the waves, in particular by unit of length and their height. The number of teeth of the two waver rollers makes it possible to adjust the number of waves.

The installation in FIGS. 13, 14 and 16 uses a waver according to FIG. 4B having the following dimensions:
“Pen” is between 0.5 and 3.5 mm, preferably between 1 and 3.1 mm, in particular approximately 2.8 mm. “R2” is between 0.1 and 5 degrees, preferably between 0.2 and 4 degrees, in particular approximately 0.50 degrees. “P” is between 0.01 and 3 mm, preferably between 0.02 and 1 mm, in particular approximately 0.08 mm. “R” is between 0.1 and 5 degrees, preferably between 0.1 and 3 degrees, in particular approximately 0.3 degrees. “Ep2” is between 0.3 and 3 mm, preferably between 0.4 and 1 mm, in particular approximately 0.68 mm. “PAS” is between 1 and 4 mm, preferably between 1.5 and 3 mm, in particular approximately 2.20 mm. “H” is between 1 and 10 mm, preferably between 2 and 5 mm, in particular approximately 4 mm. “Ang2” is between 1 and 10 degrees, preferably between 1.5 and 7 degrees, in particular approximately 3.9 degrees. “Ep1” is between 0.5 and 6 mm, preferably between 0.7 and 4 mm, in particular approximately 1.12 mm. “Ang1” is between 0.1 and 2 degrees, preferably between 0.1 and 0.75 degrees, in particular approximately 0.25 degrees.

The installation in FIG. 14 is substantially identical to that in FIG. 13, the difference between the two being the positioning of the support roller 305 of the two waver rollers, said support roller remaining at a distance from each of the two second lamination rollers 303, 304.

In FIG. 15, two lamination rollers 401 and 402 define a lamination gap, between which a first nonwoven layer NT1 is passed, on which, at a first coating point, Coating 1, lines of glue have been deposited by means of a foil, and a second nonwoven layer NT2 on which, at a second coating point, Coating 2, lines of glue have been deposited by means of a foil. Two elastic films are also passed into the gap side-by-side and at a distance from one another, between the two nonwoven layers NT1 and NT2, in order to thus bring about, in the lamination gap, their fixing to one another by lamination to the two nonwoven layers.

Upstream of the gap and of each of the coating points, each respective nonwoven layer is passed into a wave-formation gap, known as a waver or tamper, formed of two toothed rollers, referred to as waver or tamper rollers, the teeth of which interpenetrate without contact in CD as shown in FIG. 4A, which go on to shape the two nonwoven layers into the shape of waves, before they are introduced into the lamination gap, in order to thus obtain the final laminate at the exit from the gap. The relative positions of the waver rollers make it possible to adjust the shape of the waves, in particular by unit of length and their height. The number of teeth of the two waver rollers makes it possible to adjust the number of waves. The two rollers in FIG. 4A used in FIG. 15 have dimensions “Pen” of between 3 and 6 mm, preferably between 3 and 4 mm, in particular approximately 3.5 mm; “Diam” of between 20 and 50 mm, preferably between 25 and 45 mm, in particular approximately 38 mm; “R” of between 0.1 and 0.7 mm, preferably between 0.15 and 0.6 mm, in particular approximately 0.51 mm; “PAS” of between 1 and 6 mm, preferably between 2 and 5 mm, in particular approximately 4.32 mm; “Ep” of between 0.5 and 3 mm, preferably between 1.2 and 2.3 mm, in particular approximately 1.91 mm; and “d1” of between 1.5 and 4 mm, preferably between 1.7 and 3 mm, in particular approximately 2.41 mm.

In the installation in FIG. 15, after obtaining the laminate, and optionally, an activation point is also provided, in which two toothed rollers ensure transverse stretching of the laminate in order to “activate” the laminate, i.e. increase its stretching capacity by “stretching” the nonwovens that are not elastic or only slightly elastic, and/or the elastic films, which releases the elasticity of the elastics in the laminate and thus increases the elasticity thereof.

In FIG. 16, two lamination rollers 501 and 502 define a lamination gap, between which a first nonwoven layer NT1 is passed, on which, at a first coating point, Coating 1, lines of glue have been deposited by means of a foil, and a second nonwoven layer NT2 on which, at a second coating point, Coating 2, lines of glue have been deposited by means of a foil. Two elastic films are also passed into the gap side-by-side, between the two nonwoven layers, NT1 and NT2, in order to thus bring about, in the lamination gap, their fixing to one another by lamination to the two nonwoven layers.

Upstream of the gap and of one, here Coating 2, of the two coating points, the corresponding nonwoven layer, NT2, is passed into a wave-formation gap, known as a waver, formed of two toothed rollers, referred to as waver rollers, the teeth of which interpenetrate without contact in CD as shown in FIG. 4, which go on to shape the nonwoven layer into the shape of waves, before it is introduced into the lamination gap, in order to thus obtain the final laminate at the exit from the gap. The relative positions of the waver rollers make it possible to adjust the number of waves, in particular by unit of length, and their height.

According to an option of the installation in FIG. 16, it is conceivable to implement the wave formation directly on one 502 of the two lamination rollers, by providing that said lamination roller 502 forms, together with an optional waver roller 505, a wave-forming gap, either in addition to that already provided upstream, or in place thereof.

FIG. 17 shows an example of a curve showing the force as a function of the extension of the laminate having a braking effect. A braking effect is characterised by the presence of a stop, i.e. a sudden slope break between a progressive extension up to a given extension (in FIG. 18A-C, the elongation 10N) without damaging one or more components of the laminate, and then a steep slope. Said stop indicates, to the user, the best range in which the product is to be used.

The brake is defined by the presence of at least three successive slope variations, an initiation slope, a working slope, and a stop slope. The usage slope is of a lesser (positive) inclination, but not zero, than the (positive) inclination of the initiation and stop slopes.

In particular, according to the invention, the curve has an inflection point that separates a first lower curve segment having its concavity turned downwards, and a second upper curve segment having its concavity turned upwards. The first lower segment has a lower apex point, and the second upper segment has an upper apex point. The portion of the curve between the origin (coordinates F=0 for a percentage (%) of zero extension) and the lower apex point is the initiation segment of the curve, the portion of the curve between the lower apex point and the inflection point is the usage segment of the curve, and the portion of the curve between the inflection point and the upper apex point is the stop segment of the curve. It is possible to define the initiation slope as being the average slope of the initiation segment, the usage slope as being the average slope of the usage segment, and the stop slope as being the average slope of the stop segment.

In the case of the laminates of the prior art, there is no progressive counterreaction of the hand in the usage zone, and the user enters the stop zone directly, without any transition, such that they confuse it with the usage zone.

The usage slope according to the invention is preferably positioned before a 100% extension. The start of the usage region according to the invention is preferably positioned at a force greater than 2 N. The end of the usage region according to the invention is preferably positioned at a force of less than 10 N.

In particular, the X-axis of the initiation segment extends between 0% and a value of between 5% and 20%, for example 10%, and the X-axis of the usage segment extends between a value of between 5% and 20%, for example 10%, and a value of between 60% and 80%, for example 70%, while the X-axis of the stop segment extends, following the usage segment, up to a value of between 80% and 120%, in particular, as mentioned in the paragraph above, 100%.

FIGS. 18A, 18B and 18C show examples of curves for the examples of laminates according to the invention.

Hereinafter, three examples of laminates according to the invention, denoted E46, E53 and HG1, respectively, are described.

### Example 1: HG1

The upper nonwoven is a nonwoven available from SANDLER under the reference SPUNLACE SAWASOFT 2925 at 30 g/m². The elastic film formed, based on SIS, is extruded with an average basis weight of 50 g/m², the lower nonwoven being a nonwoven available from FITESA under the reference SPUNBOND HES CD Rod with a basis weight of 20 g/m². The structure of example 1 is as shown in FIG. 1b. The product has been obtained according to the method shown in FIG. 15 and using the waver in FIG. 4A, with a blade penetration of between 3 and 4 mm.

### Example 2: E46

The upper nonwoven is a nonwoven available from SANDLER under the reference SPUNLACE SAWASOFT 2626 at 25 g/m². The elastic film formed, based on SIS 50, is extruded with an average basis weight of 50 g/m². The lower nonwoven is a nonwoven available from Texbond SPa under the reference SPUNBOND ULTRASOFT with a basis weight of 18 g/m². The structure of example 2 is as shown in FIG. 1. It comprises 17 waves obtained according to the method shown schematically in FIG. 14 and using the waver in FIG. 4B, with a blade penetration of approximately 2.8 mm.

Example 3: E53

Example 3 is similar to example 2, the difference being that the penetration airgap is in the region of 2.7 mm for example 3, instead of 2.8 mm for example 2.

1. An elastic laminate, extending widthwise in a first direction, specifically CD, and lengthwise in a second direction, specifically MD, comprising:
at least one nonwoven layer; and
at least one elastic film fixed by an upper face or lower face, respectively, to a lower face or upper face, respectively, of the at least one nonwoven layer;
at least one adhesive layer being provided between the at least one nonwoven layer and the at least one elastic film,
characterised in that
when the laminate is not in the stretched state, the nonwoven layer comprises, in transverse cross-section according to the first direction, specifically in the transverse cross-section CD, curved sections in an inverted U, specifically in the shape of a Ω, separated from each other by intermediate sections,
the intermediate sections are in contact with the at least one adhesive layer, while the curved sections are not in contact with the at least one adhesive layer.

2. The elastic laminate, extending widthwise in a first direction, specifically CD, and lengthwise in a second direction, specifically MD, comprising:
at least one nonwoven layer; and
at least one elastic film fixed by an upper face or lower face, respectively, to a lower face or upper face, respectively, of the at least one nonwoven layer;
the at least one nonwoven layer being fixed to the at least one elastic film in fixing zones, in particular by ultrasonic welding, calendering or laminating,
characterised in that
when the laminate is not in the stretched state, the nonwoven layer comprises, in transverse cross-section according to the first direction, specifically in the transverse cross-section CD, curved sections in an inverted U, specifically in the shape of a Ω, separated from each other by intermediate sections,
the intermediate sections being in contact with the at least one elastic film, while the curved sections are not in contact with the at least one elastic film.

3. The elastic laminate according to claim 1, characterised in that the nonwoven layer or layers forming the at least one nonwoven layer extend(s) in the first direction over a total nonwoven width, in particular in the direction CD, and the elastic film or films forming the at least one elastic film extend(s) over a total elastic width, the ratio equal to the total elastic width to the total nonwoven width being between 0.3 and 0.9, preferably between 0.4 and 0.8.

4. The elastic laminate according to claim 1, characterised in that the curved sections extend in the first direction over a total curved section width, in particular in the direction CD, and the intermediate sections extend over a total intermediate section width, the ratio equal to the total curved section width to the total width of the nonwoven being between 0.25 and 0.85, preferably between 0.35 and 0.75.

5. Elastic laminate according to claim 1, the laminate having an initial thickness (e₀) before its first stretching, and when the laminate is stretched according to the first or the second direction up to a so-called maximum elongation, at least equal to 50%, the curve giving the relative loss of thickness [(e₀−e(t))/e₀] as a function of the elongation (t) has a maximum value of between 5 and 50%, preferably less than or equal to 40%, even more preferably less than 35%.

6. The elastic laminate according to claim 5, characterised in that the ratio of the initial thickness (e₀) minus the thickness (eₘₐₓ) of the laminate at maximum elongation over the initial thickness (e₀), i.e. the ratio [(e₀−eₘₐₓ)/e₀], is less than or equal to 55%, in particular less than 40%, in particular less than 35%, and/or is greater than 5%, in particular greater than 10%, in particular greater than 20%.

7. The elastic laminate according to either claim 5, characterised in that the ratio of the initial thickness (e₀) minus the thickness (e₁₀₀) of the laminate at 100% elongation over the initial thickness (e₀), i.e. the ratio [(e₀−e₁₀₀)/e₀], is less than or equal to 55%, in particular less than 40%, in particular less than 35%, and/or is greater than 5%, in particular greater than 10%, in particular greater than 20%.

8. The elastic laminate according to claim 1, characterised in that the laminate, after stretching to an elongation of 100% proceeding from the first non-stretched state and then relaxation to a second non-stretched state (0% elongation), has a thickness (eᵥ), and the ratio of the thickness (eᵥ) minus the thickness (e₁₀₀) of the laminate at 100% elongation over the thickness, i.e. the ratio [(eᵥ−e₁₀₀)/eᵥ], is less than or equal to 60%, in particular less than 50%, in particular less than 40%, in particular less than 35%.

9. (canceled)

10. (canceled)

11. (canceled)

12. (canceled)

13. The elastic laminate according to claim 1, characterised in that, after stretching to an elongation of 100% proceeding from the initial state, i.e. before its first stretching, and then relaxation to the non-stretched state (0% elongation), it has a thickness (eᵥ) greater than the initial thickness (e₀), and the ratio [(eᵥ−e₀)/e₀] is greater than 2%, in particular greater than 5%, more particularly greater than 10%, and is preferably less than 70%, in particular less than 50%, more particularly less than 35%.

14. The elastic laminate according to claim 1, characterised in that, in the non-stretched state of the laminate and/or of the elastic film, the nonwoven layer comprises, alternately, in the transverse cross-section CD, intermediate sections, in particular straight or substantially straight, in particular curved having their concavity turned upwards with less curvature than that of the inverted U shapes, extending in the direction CD being fixed to the elastic film, and inverted U-shaped curved sections, in particular in the shape of a Ω, each consisting of two strand sections proceeding from two successive intermediate sections and meeting at an apex, which are not fixed to the elastic film and remain at a distance therefrom, so as to define empty spaces between them and the elastic film.

15. (canceled)

16. (canceled)

17. (canceled)

18. (canceled)

19. The elastic laminate according to claim 1, characterised in that it is provided with an additional nonwoven layer, on the other side of the elastic film.

20. (canceled)

21. The elastic laminate according to either claim 19, characterised in that the nonwoven layer has a first basis weight and the additional nonwoven layer has a second basis weight which is different from the first basis weight, in particular greater than the first basis weight.

22. (canceled)

23. (canceled)

24. A method for manufacturing a laminate.
the method comprising the steps in which:
an elastic film is taken;
a nonwoven layer, in particular a nonwoven having continuous filaments, in particular a spunbond, is taken;
the nonwoven layer is shaped to obtain a nonwoven layer in the form of waves having, in transverse cross-section, the form of a succession of inverted U shapes, in particular in the shape of a Ω, which are separated by intermediate sections, in particular straight or substantially straight, or curved or substantially curved, having their concavity turned upwards, having less of a curvature than that of the inverted U shapes; and
the nonwoven layer in the form of waves is fixed to the elastic film, in particular at the side of the nonwoven layer opposite the crests of the waves.

25. A nappy for a baby or adult incontinence pants comprising at least one elastic laminate according to claim 1.

26. An elastic laminate with hooks, comprising an elastic laminate according to claim 1 and at least one lap with hooks fixed to the elastic laminate, in particular on the nonwoven layer, preferably in a zone without a wave or inverted U.

27. A roll comprising a laminate according to claim 1, said laminate having a length at least greater than 1 metre and being rolled according to an axis perpendicular to the axis MD, said laminate comprising two adjacent elastic films.

28. (canceled)

29. The elastic laminate according to claim 2, characterised in that the nonwoven layer or layers forming the at least one nonwoven layer extend(s) in the first direction over a total nonwoven width, in particular in the direction CD, and the elastic film or films forming the at least one elastic film extend(s) over a total elastic width, the ratio equal to the total elastic width to the total nonwoven width being between 0.3 and 0.9, preferably between 0.4 and 0.8.

30. The elastic laminate according to claim 2, characterised in that the curved sections extend in the first direction over a total curved section width, in particular in the direction CD, and the intermediate sections extend over a total intermediate section width, the ratio equal to the total curved section width to the total width of the nonwoven being between 0.25 and 0.85, preferably between 0.35 and 0.75.

31. The elastic laminate according to claim 2, the laminate having an initial thickness (e₀) before its first stretching, and when the laminate is stretched according to the first or the second direction up to a so-called maximum elongation, at least equal to 50%, the curve giving the relative loss of thickness [(e₀−e(t))/e₀] as a function of the elongation (t) has a maximum value of between 5 and 50%, preferably less than or equal to 40%, even more preferably less than 35%.

32. The elastic laminate according to claim 2, characterised in that, in the non-stretched state of the laminate and/or of the elastic film, the nonwoven layer comprises, alternately, in the transverse cross-section CD, intermediate sections, in particular straight or substantially straight, in particular curved having their concavity turned upwards with less curvature than that of the inverted U shapes, extending in the direction CD being fixed to the elastic film, and inverted U-shaped curved sections, in particular in the shape of a Ω, each consisting of two strand sections proceeding from two successive intermediate sections and meeting at an apex, which are not fixed to the elastic film and remain at a distance therefrom, so as to define empty spaces between them and the elastic film.

33. The elastic laminate according to claim 2, characterised in that it is provided with an additional nonwoven layer, on the other side of the elastic film.

34. The elastic laminate according to either claim 33, characterised in that the nonwoven layer has a first basis weight and the additional nonwoven layer has a second basis weight which is different from the first basis weight, in particular greater than the first basis weight.

35. A nappy for a baby or adult incontinence pants comprising at least one laminate according to claim 2.

36. An elastic laminate with hooks, comprising an elastic laminate according to claim 2 and at least one lap with hooks fixed to the elastic laminate, in particular on the nonwoven layer, preferably in a zone without a wave or inverted U.

37. A roll comprising an elastic laminate according to claim 2, said elastic laminate having a length at least greater than 1 metre and being rolled according to an axis perpendicular to the axis MD, said elastic laminate comprising two adjacent elastic films.
